(19) United States
(12) Patent Application Publication
(10) Pub. No.: US 2006/0025929 A1
(43) Pub. Date: Feb. 2, 2006

(54) Method of determining a genetic relationship to at least one individual in a group of famous individuals using a combination of genetic markers

(76) Inventor: Chris Eglington, London (GB)

Correspondence Address: Michelle S. Temple;Temple Patent Law Office, 57 Hackleboro Road, Canterbury, NH, 03224, US

(21) Appl. No.: 10/903,043

(22) Filed: Jul. 30, 2004

(51) Int. Cl. G06Q 10/00; G01N 33/48; G06Q 50/00

(52) U.S. Cl. 702/19; 702/20; 705/2

(57) ABSTRACT

A method for quantifying the genetic relationship of a customer to at least one individual of at least one group of famous individuals, the method includes the following steps. First, receiving genetic information and famous individual group selection criteria from a customer, next, calculating the genetic distance of the customer to at least one individual within the group and finally, reporting the results to the customer.

### FIELD OF THE INVENTION

This present invention relates to the field of genealogy and more particularly to a method of quantifying the genetic relationship to a group of famous individuals using a series of genetic markers.

### BACKGROUND OF THE INVENTION

A typical practice of genealogists is to gather information on family history, such as names, birth dates, death dates, marriages, etc., and record such information on a standard lineal branching chart, commonly known as a “pedigree chart”. Animal breeders use a similar chart to record pedigree information on horses, dogs, cats, etc. Such methods of genealogical research impose a number of inherent limitations. The most critical limitation is that without the knowledge of an individual human or animal's birth parents, it is virtually impossible to determine from which pedigree the individual descends. Following, without an individual's pedigree, using this method, it is impossible to determine to what degree an individual is related to a random or specific second individual or group of individuals.

More recently, genealogical methods have included using the Y-chromosome as a molecular marker to establish the degree of ancestry by paternal descent between two male individuals. Using this method, markers are selected on the Y-chromosome and compared between the two individuals to determine the degree of relatedness.

Alternatively, mitochondrial DNA (mtDNA) can be used for both male and female individuals to determine the degree of maternal relationship with a particular woman. Similarly to the Y-polymorphism method, particular markers are compared between the two individuals to determine the degree of relatedness. However, both these methods are limited in that they assess the genetic history of a discrete part of the genome (Y-chromosome or mtDNA) and not of the genome as a whole.

Given the current demand in the art of completing genealogical studies by both completing each individual family pedigree and linking each family pedigree to one common ancestor, a method for finding previously unknown individuals that are related to a particular pedigree is highly desired. In particular, an approach for quantifying the degree of relatedness of the genome of a particular individual to the genome of any one of a random collection of individuals is highly desired.

Given the interest in famous figures, either historical or contemporary, a method for determining to what degree a particular individual is related to a famous individual is highly desired.

Therefore, what is needed in the art is a method for quantifying the degree of genetic relationship between a particular individual and any one of a collection of individuals, or a famous individual, using a set of genetic markers. A method of generating a “genomic pedigree”, that is, one based only on genetic markers, is further desired.

### SUMMARY OF THE INVENTION

In accordance with one aspect of the invention, a method for quantifying the genetic relationship of a customer to at least one individual of at least one group of famous individuals, the method includes the following steps. First, receiving genetic information and famous individual group selection criteria from a customer, next, calculating the genetic distance of the customer to at least one individual within the group and finally, reporting the results to the customer.

Implementation of this aspect of the invention may include one or more of the following features: where the genetic information is a biological sample provided by the consumer, where the genetic information is data provided by the consumer. Also, the method may include, as part of the step of calculating the genetic distance of the customer to at least one individual within the group, the determination of the proportion of a control population that is more closely or less closely genetically related to the at least one famous individual as compared with the customer. The reporting of the results to the customer may include ranking the order of most closely genetically related famous individuals to the customer.

In accordance with another aspect of the invention, a system for quantifying the genetic relationship of a customer to at least one individual of at least one group of famous individuals, the system including one or more computers operably programmed and configured to perform the following steps. First, receive genetic information and selection input, the input selecting at least one group of famous individuals. Second, process the genetic information and selection input to calculate a first genetic distance value between the genetic information received and each famous individual within the group. Finally, display a result of the calculation, whereby the result is a list of the famous individuals and a corresponding degree of genetic relationship between each famous individual and the customer.

Implementation of this aspect of the invention may include one or more of the following features. The system may include ranking the order of the genetic distance. The system may include displaying the result via an Internet website, and displaying the result via computer monitor. The system may include: (1) processing the genetic information, (2) calculating a second genetic distance value between a control population and each famous individual within the group; and (3) comparing the second genetic distance values with the first genetic distance value to yield a relationship value that takes into account the degree of genetic heterogeneity in the populations from which the test individuals come.

These aspects of the invention are not meant to be exclusive and other features, aspects, and advantages of the present invention will be readily apparent to those of ordinary skill in the art when read in conjunction with the appended claims and accompanying drawings.

### BRIEF DESCRIPTION OF THE DRAWINGS

FIG. 1 is a diagram depicting the functional overview of the present invention;

FIG. 2 is a flow diagram representing the functional overview of the present invention;

FIG. 3 is a diagram depicting the functional overview of the haplotypic comparison;

FIG. 4 is a table representing example genotypes of three unrelated Canadians using the Profiler system;

FIG. 5 is a diagram depicting the functional overview of the haplotypic comparison using the control database; and

FIG. 6 is a block diagram of a computer system upon which embodiments of the invention may be implemented.

### DETAILED DESCRIPTION OF THE INVENTION

In the following description, for the purposes of explanation, specific details are set forth in order to provide a thorough understanding of the invention. However, it will be apparent that the invention may be practiced without those specific details. In other instances, well-known structures and devices are depicted in block diagram form in order to avoid unnecessarily obscuring the invention.

Various aspects and features of example embodiments of the invention are described in more detail hereinafter in the following sections: (1) functional overview; (2) biological sample; (3) group criteria; (4) famous persons group database; (5) control population database; (6) genetic distance algorithm; and (7) implementation mechanisms.

### 1. Functional Overview

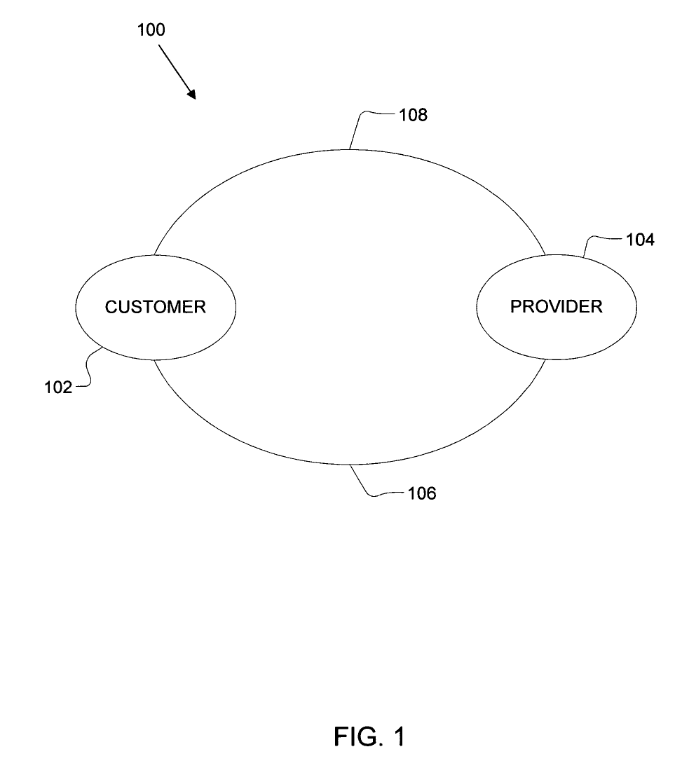

FIG. 1 is a block diagram 100 that illustrates an overview of the approach for determining the degree of genetic relationship between an individual and at least one group of famous persons using a genetic marker system to determine the genetic distance between the individual and one or each member of each group of famous persons selected. As used herein, the term “genetic marker” refers to a polymorphic region of the human genome, a “genotype” is defined as the genetic constitution at a discrete genetic marker of the individual, and “haplotype” is defined as the combination of genotypes across a series of genetic markers for a given individual. Customers can be located anywhere in the world, and the provider can be located anywhere in the world.

According to one embodiment, a customer 102 provides a biological sample and a selection of group criteria in which they wish to have their haplotype compared. The customer 102 sends the sample over a delivery channel 108 and selection criteria over a link 106 to the provider 104. Link 106 may be any medium for transferring data between customer 102 and provider 104 and the invention is not limited to any particular medium. Examples of link 106 include, without limitation, a network such as a LAN, WAN or the Internet, a telecommunications link, a wire or optical link, a wireless connection or any physical delivery channel including, without limitation, mail delivery, courier delivery or delivery using a delivery agent. The delivery channel 108 includes any method of delivery of a sample including, without limitation, a network such as a LAN, WAN or the Internet, a telecommunications link, a wire or optical link, a wireless connection or any physical delivery channel including, without limitation, mail delivery, courier delivery or delivery using a delivery agent. In some cases, both the sample and the selection criteria are sent over the delivery channel 108. In other cases, the sample information is and the selection criteria are sent over the link 106. Provider 104 may be centralized or distributed.

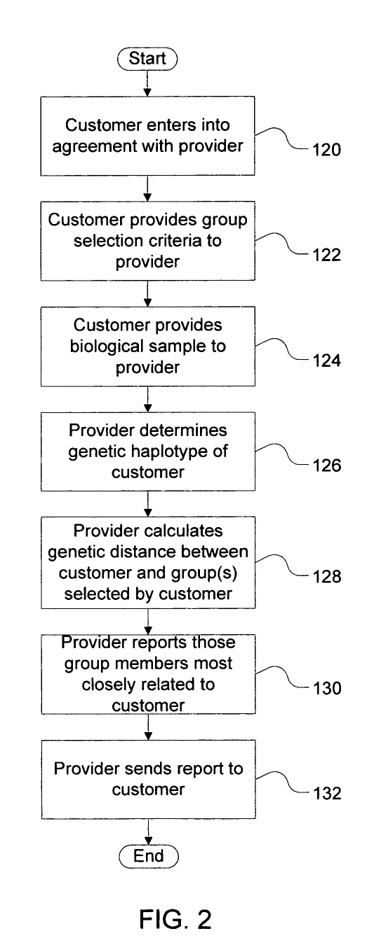

Referring next to FIG. 2, one embodiment of the invention is shown. In step one 120, the customer enters into an agreement with the provider. Next, in step 122, the customer provides group selection criteria to the provider. In step 124, the customer provides a biological sample to the provider. The provider determines the haplotype of the sample in step 126 and then, in step 128, the provider measures the genetic distance between the customer and the group criteria. In step 130, the provider reports those group members most closely related to the customer. The provider in step 132 then sends this report to the customer.

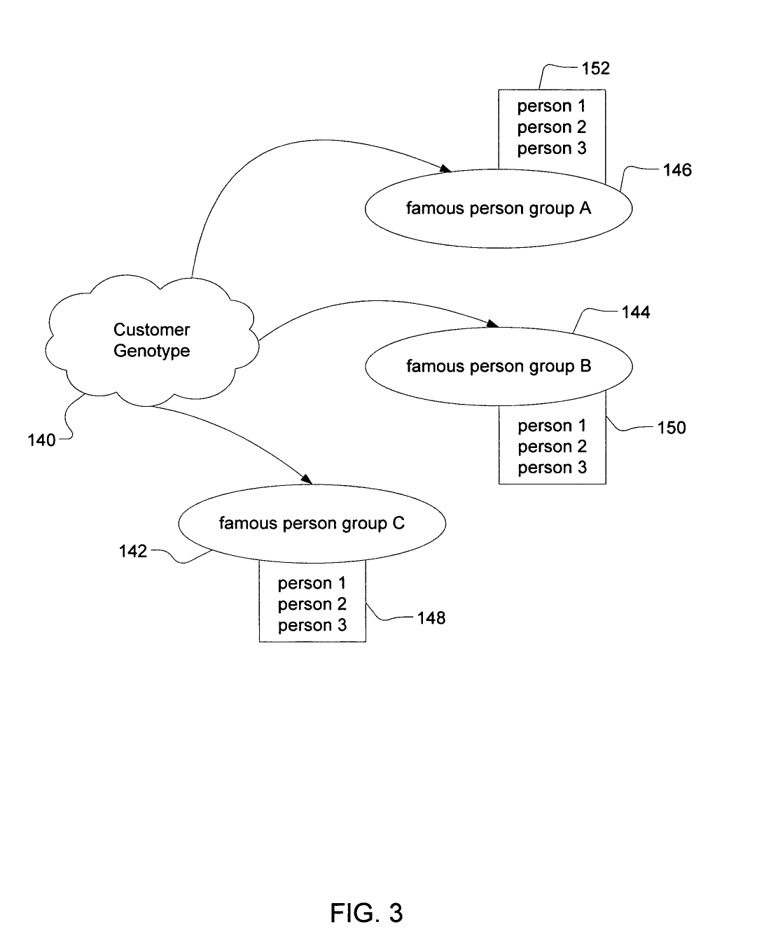

Referring next to FIG. 3, each individual customer genotype 140 is compared to each group specified by the customer in the group selection criteria 142, 144, 146. Within each famous person group 142, 144, 146, the customer's genotypes are compared individually to each person's within that group 148, 150, 152. Results are categorized according to the degree of genetic relationship as determined by the combination of genetic markers employed. Those famous persons most closely related genetically, as determined by the system employed here, to the customer are called “Famous Person Candidates”. Both the names of the famous person candidates, and the degree of relationship are reported to the customer.

### 2. Biological Sample

The customer 102 provides a biological sample to the provider 104. The biological sample is either a physical sample or a data sample.

In the preferred embodiment, the customer 102 contacts the provider 104 either through the link 106 or through a delivery system 108. In response, the provider 104 either sends the customer 102 a sample-harvesting packet or describes the data needed from the customer 102. The customer 102 indicates a preference. If a sample-harvesting packet is requested, the provider 104 sends this through the delivery channel 108. If the customer 102 indicates they prefer to send data, the customer 102 can either send the data through the link 106 or the delivery channel 108.

The sample-harvesting packet can be any type of biological sample collection kit that collects a sample that can be genotyped. A customer 102 can use the sample-harvesting kit properly without requiring medical assistance. The types of kits include, without limitation, any system used to collect cells from an individual for the purpose of DNA extraction. Typically this would be a cheek/mouth swab kit, a hair sample collection kit, a blood sample collection kit or a skin sample collection kit.

Additionally, the customer can send in any biological sample that contains DNA, including, without limitation, bone, teeth, mouth wash and blood.

The data sample is provided to the provider 104 either electronically, through the link 106, or physically over the delivery channel 108. The data sample can be provided by customers 102 who are in possession of the genetic marker information required by the provider 104.

Once the provider 104 receives the sample-harvesting kit, or the biological sample, the provider 104 processes the kit to determine the genetic information for any genetic marker, which will then be used to determine the genetic relationship between the customer 102, and each person in each of the group criteria selected.

Referring again to FIG. 2, a flow diagram of one embodiment of the overall process is shown. The customer enters into an agreement with the provider 120, which includes specific information about privacy and payment, among other terms. The agreement can be entered into by email, or on the Internet. The agreement can also be entered into via electronic documents, where the customer signs using an electronic signature, or, the agreement can be sent via the mail and the customer signs and returns the agreement to the provider.

Next, the customer provides group selection criteria to the provider 122. The group selection criteria specify the groups of famous persons in which the customer wishes to have their haplotype compared. The selection can be provided either over the Internet, using selection pages, or via electronic mail. Additionally, the customer could provide the selection criteria using a paper selection process or calling the provider and verbally giving the selection criteria to the provider. The customer can select any number of groups of selection criteria.

Next, the customer provides a biological sample to the provider 124. As described above, the biological sample can be either a specimen or data. Next, the provider calculates the genetic distance between the customer and the group(s) selected by the customer. A comparison is made between the customer and each of the persons in each of the groups selected by the customer. Examples of comparison algorithms are described below.

The provider then reports those group members most closely related to the customer 130. In other embodiments, the provider reports a percentage relation of the customer to each person in each of the groups selected. In other embodiments, an additional analysis is done, where the provider compares the relation of the customer to each of the persons in the groups selected to the relation of a control population to each of the persons in the groups selected. The reports are sent either via electronic mail or regular mail as described above (and shown in FIG. 1).

### 3. Group Criteria

The group criteria indicate the group or groups of famous persons in which the customer 102 desires to have their haplotype compared. The group criteria can be selected by the customer 102 on a web page or via an email and sent over the link 106, or selected on a form sent over the delivery channel 108. The customer 102 selects at least one group of famous people, the groups including, without limit, US Presidents, Founding Fathers, Royal Families from various nations, Baseball Players, Football Players, Rock Stars, Actors and Actresses, Hockey Players, Authors, Artists and Scientists. The groups can have any number of members, and can be a complete group or an incomplete group to any degree. The invention contemplates any number of groups and is not limited in any way to a specific number or the groups enumerated above. Additionally, one individual can be a member of various groups. For example, John F. Kennedy can be a member of the Irish Descendants group and US Presidents.

In one embodiment, the customer selects one particular individual in the group for comparison.

### 4. Famous Persons Group Database

The group database is a database that has been programmed with the genotypic information of each member of each group of famous people. The group database is used to calculate the degree of genetic relationship between the customer and each person in the selection of group criteria. The database is created using any method known in the art for obtaining DNA information of an individual. These methods include, without limit: (1) obtaining DNA directly from the individual or (2) in the case of certain uniparentally inherited genetic systems (e.g. Y-chromosome and mtDNA) inferred by examining DNA of direct descendants of each individual (i.e. the DNA of male descendants for Y-chromosome information or from ancestors along the maternal lineage for mtDNA).

### 5. Control Population Database

The control population database is a database containing published data sets of large populations from different geographical locations. The control population database, in the preferred embodiment, is compiled using published forensic databases available on the Internet. In other embodiments, the control population database is any database containing the genetic data of a large number of individuals.

### 6. Genetic Distance Algorithm

The process used to determine the famous person candidates, or the persons most closely related to the customer, is performed using a genetic distance algorithm. Genetic distance is a measurement of the overall relationship between two individuals, or as taken another way, the sum of all the different ways in which two individuals are related. Genetic distance is a way of measuring the amount of evolutionary divergence between two individuals, or populations, of a species by quantifying the amount of genetic divergence occurring between individuals or populations. Genetic distance can be calculated by a number of methods.

A number between 0 and 1 can be used to represent the genetic distance between two individuals. A genetic distance of 0 indicates that the two individuals are genetically identical, such as would be the case with identical twins (monozygotic). In comparison, a genetic distance of 1 would indicate a much more distant relationship between the individuals. Genetic distance can also be represented in other forms. For example: as the percentage of a population who were more closely/less closely related; as a description of the likely relationship, for example, “shares a similar level of relatedness as a 5th Cousin”; and as the time to most recent common ancestor (MRCA). While genetic distance can be used to compare populations, it can also be used as a measure between two people. Genetic distance can be extended to identify to which person among a group of persons (for example, US Presidents or the members of a sports team) an individual is most closely genetically related.

An example of how genetic distance relates to the relatedness of one individual to another is as follows. Using two random Englishmen, they will be related if we look far enough into the past. For example, if their MRCA lived in AD 1400, about 20 generations ago, they are 21st cousins. However, the two individuals will also share more ancestors further in the past on different sides of the family. A genetic distance represents the sum of all these far-off shared ancestries.

Referring now to FIG. 3, each individual genotype of the customer 140 is compared to each of persons 148, 150, 152 within the group of famous persons selected 142, 144, 146 under the group criteria. The comparison is done by calculating the genetic distance between the customer 140 and each individual 148, 150, 152 within the famous person groups 142, 144, 146 selected. Each person in each group of famous persons selected is ranked in order of their genetic distance to the customer to identify which group members are most closely genetically related to the customer.

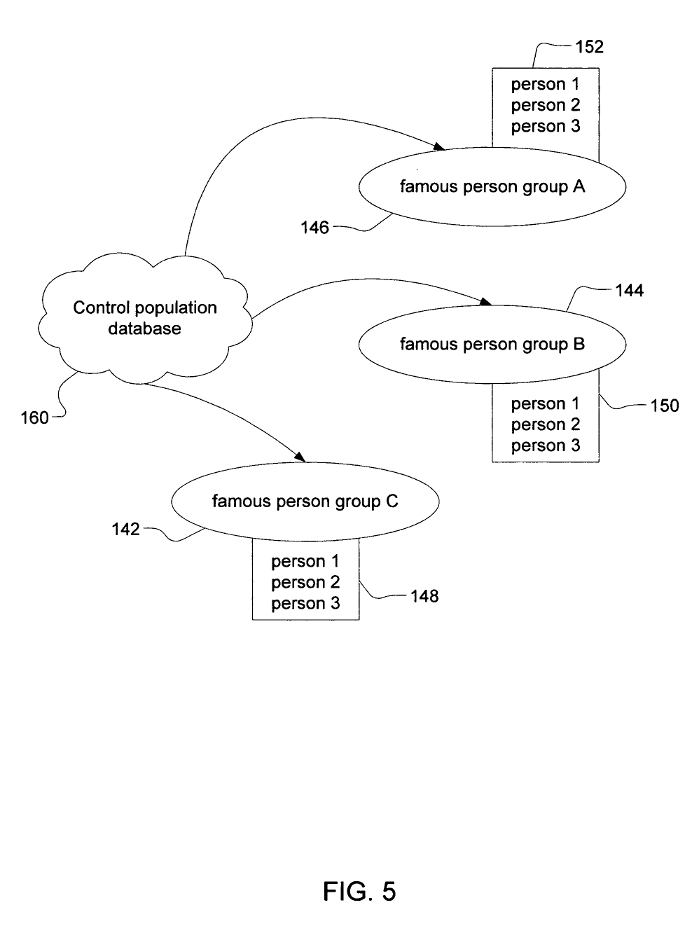

Referring next to FIG. 5, genotypes of each individual in the control population database 160 are compared to each person 148, 150, 152 within the group of famous persons selected 142, 144, 146 under the group criteria. The comparison is done by calculating the genetic distance between each individual genotype in the control population 160 to each individual 148, 150, 152 within the famous person groups 142, 144, 146 selected.

A comparison calculation is performed between each individual in the control population compared with the customer. The genetic distances are compared and a relationship value is determined. The relationship value is the proportion of the control population that is more closely related and less closely related to each of the famous persons as compared with the customer.

### Generic Algorithm

The following is an example of one of the generic (sic) algorithms that are the basis for all genetic distance algorithms and is customized based on the types of markers used. Any genetic distance algorithm can be used for the invention, and the algorithms presented herein are given as examples only. A marker is an identifiable polymorphic region on a chromosome or mtDNA (e.g., single nucleotide polymorphism, microsatellite, variable number tandem repeat, transposon, etc.) whose inheritance can be monitored. Markers can be located anywhere in the genome or mtDNA. They can be in regions of coding DNA (genes—exonic or intronic) or some segment of DNA with no known function. Any marker or set of markers can be used in the genetic algorithm to compute genetic distance and the algorithm is modified depending on the type of markers used.

In one embodiment, microsatellite markers are used, and an appropriate distance is known as the allele-sharing distance. Microsatellite markers are short sequences of di- or trinucleotide repeats of very variable length distributed widely throughout the genome. The number of identical alleles that are shared between the two individuals is counted. An allele is a copy of a marker (for autosomal markers an individual has 2 alleles—1 from each chromosome). Individuals can share 0, 1 or 2 alleles at a given marker.

The algorithm can be represented as d=1−(n/m), where d is the allele-sharing distance, n is the actual number of shared alleles, and m is the maximum possible number of alleles that can be shared. This algorithm is called the allele-sharing distance algorithm. This algorithm can be extended by giving weight to markers proportionally to the degree of similarity between the individuals, but not actually shared between the two individuals.

Using microsatellite markers as an example in the allele-sharing distance algorithm, n would be replaced by Σw where w=1−r/x (w is the degree of sharing of an allele), r is the difference in number of repeats found for a marker when comparing two individuals, and x is the range of repeat units observed in the control population for this marker. The number of repeats refers to a DNA repeat, where a repeat is a fragment of DNA sequence, typically 2 to 4 nucleotides in length, that repeats itself to form a microsatellite marker.

In one embodiment, the genetic distance is reported to the individual as a percentage of the population. To express this genetic distance as a percentage of a population who were more closely/less closely related to the famous person the result of the genetic distance between the individual and each of the famous persons in the selected groups of famous persons are compared to a control population database, representing the general population.

The genetic distance between each individual in the control population database and each of the famous persons in the groups of famous persons selected will be calculated. A report is generated ranking each individual in the control population database in order of their genetic distance to each of the famous persons, and an indication of where the customer's results fall.

In one embodiment, the results of this calculation are expressed as an estimation of the degree of relationship in terms of first, second, third, fourth, etc cousins with each of the famous persons. This “cousin relationship” calculation will be possible by comparison to a simulated data set of x pairs of each type of cousin. A large number of unrelated people, from the control database, may be selected as the base generation. In other embodiments, the base generation is created randomly by choosing alleles to create each individual in proportion to their allele frequency in the control population.

The next generation is then generated by applying the laws of Mendelian inheritance to randomly chosen pairs of individuals in the base generation, creating a number of offspring of each pair, with one allele randomly chosen from each parent. This same process is continued by choosing to randomly ‘mate’ pairs from this generation to create the next generation, etc.

The simulation through the generation continues as described. The simulation is ended at any appropriate number of generations, for example, the simulation is ended at five generations (fourth cousins) and then the number of shared alleles between all pairs of fourth cousins is counted. This yields a distribution of genetic distances for fourth cousins. This entire process is repeated over and over to generate enough pairs of cousins, and therefore, enough data points, for a valid comparison with the empirical genetic distance, for example, that observed between each famous person and the customer.

The probabilities of the test pair being third, fourth, fifth cousins, etc. is calculated. For example, if the distribution of the genetic distances between the simulated third cousin pairs peaks at 0.55, and that for fourth cousin pairs at 0.59, then a test pair with a distance of 0.55 will have a higher probability of being third cousins than fourth. A pair with d=0.57 will have more equal probabilities and a pair with d=0.60 will be much more likely fourth cousins (in all cases depending on the exact shape of the simulated distributions). In all cases there is a higher likelihood that the test pairs are third or fourth cousins compared to the likelihood that the test pairs are not related.

Additionally, from studies of the rate of mutation for individual markers it is possible to estimate when two individuals shared their most recent common ancestor by counting the number of identical markers these individuals share.

With a sufficiently large database of genotypes, it is possible to estimate the proportion of the population that is more closely or less closely related to the famous individual compared to the relationship of the test individual from the control population. The genetic distance from each individual in the control population database is calculated and then ranked in order of their genetic distance to each of the famous persons. A comparison can then be done with genetic distance results from the customer.

The following are examples of three algorithms that can be employed to calculate the genetic distance. Although examples of algorithms are given herein, many different methods can be used to calculate interindividual variation. The invention is intended to include any algorithm for calculating the variation, and is not limited to the algorithms described herein.
1) D_{SA} Distance Between Shared Alleles

$${D}_{SAI} = 1 - {P}_{SAI} , with , {P}_{SAI} = \frac{{}_{i}^{r} S}{\overline{2} r}$$

where the number of shared alleles S is summed over all loci r.
2) Dfs, Fuzzy Set Similarity

The fuzzy set similarity between 2 individuals is the ratio between the cardinality of the intersection of their alleles and the cardinality of the union of their alleles, e.g., if two individuals have genotypes ab and ac, the intersection is {a}, the union is {a,b,c}, and the ratio is 1/3.

The distance can be taken as: Dfs=−ln(fs), or Dfs′=1−fs

3) D₁ Statistic

Inter-individual genetic similarity can be estimated according to:

I=_XiYi/(_Xi²_Yi)^{1/2}, where Xi and Yi is the frequency of i th allele for each locus in the individual X and Y, respectively. If individual X has genotype AA at locus L, the frequency of allele A at that locus in the individual X was defined as A=1.0; if individual X had genotype AB at that locus, the frequency of allele A and B in the individual X was defined as A=0.5 and B=0.5.

A pairwise genetic distance measure can be calculated as D₁=(1−Ik), where Ik is the average of the I values calculated for each locus.

### EXAMPLE

### Example of Allege-Sharing Distance Algorithm

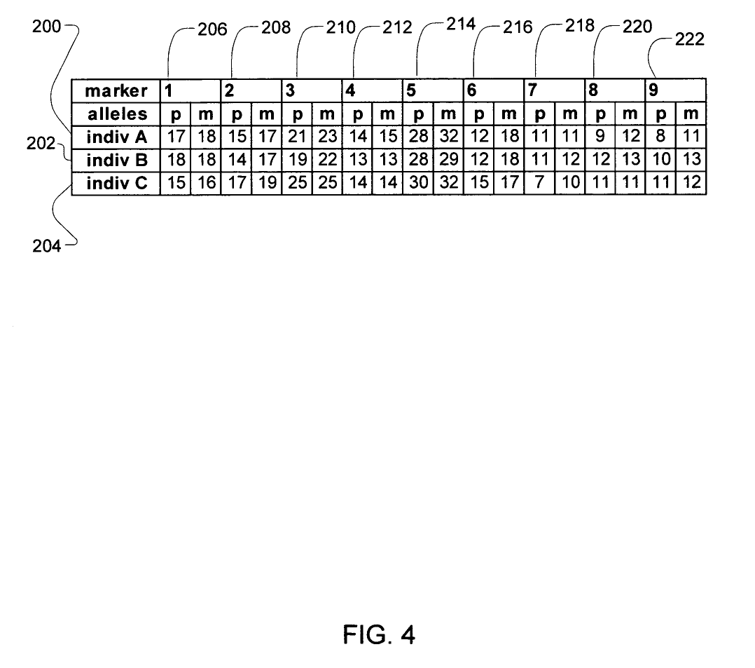

FIG. 4 is a table of example genotypes of three unrelated Canadians, individual A 200, B 202, and C 204 using the Profiler system (from Police database online). There are nine microsatellite markers 206-222, each with two copies or alleles per individual, the paternal (p) and maternal (m). The actual genotypes in the array consist of the numbers of repeats of the base unit at each microsatellite marker.

The number of alleles shared between the two individuals is first counted. Individuals can share 0, 1 or 2 alleles at a given marker. In this example the overall number shared can thus range from 0 to 18. For these three, A 200 and B 202 share 7, A 200 and C 204 share 4 and B 202 and C 204 share only 1 allele. For comparison with other data sets which may have different numbers of markers, the proportion of shared alleles is then calculated, so A 200 and B 202 share 7/18=0.39, A 200 and C 204=0.22 and B 202 and C 204=0.06. Finally this number (a similarity) is subtracted from one to give a distance, for A 200 and B 202, d=0.61, for A 200 and C 204, d=0.78 and for B 202 and C 204, d=0.94. The formula is d=1−p, where d is the allele-sharing distance and p is the proportion of shared alleles.

As demonstrated with this example, people that are not closely related can have very variable genetic distances, some are obviously more distantly related than others. In this case individual C 204 is actually Native American, which may explain his somewhat greater distance from A 200 and B 202 who are Caucasian. Note also that A 200 and B 202, even though they are unrelated, share a number of alleles due to their shared European heritage, or shared ancestors deep in the past. This background sharing is also due to the fact that there are only a finite number of alleles at each marker, therefore two individuals may have the same allele at a marker even though they did not inherit it from a common ancestor at some point in the past.

Alleles can mutate from one into another. A study of such sharing among Italians using these same markers shows that the average pair of unrelated individuals shares 5 alleles (varies from 0 to 12), while for instance full brothers share on average 11 (range from 6 to 16). See Presciuttini et al (2003) Forensic Science International 131: 85-89. A much larger number of markers will be required to give an accurate estimation of cousin relationships, where less than half the alleles will be shared, but the number of alleles shared is more than the number shared by unrelated individuals. Following, using a greater number of markers gives a more accurate degree of sharing.

### 7. Implementation Mechanisms

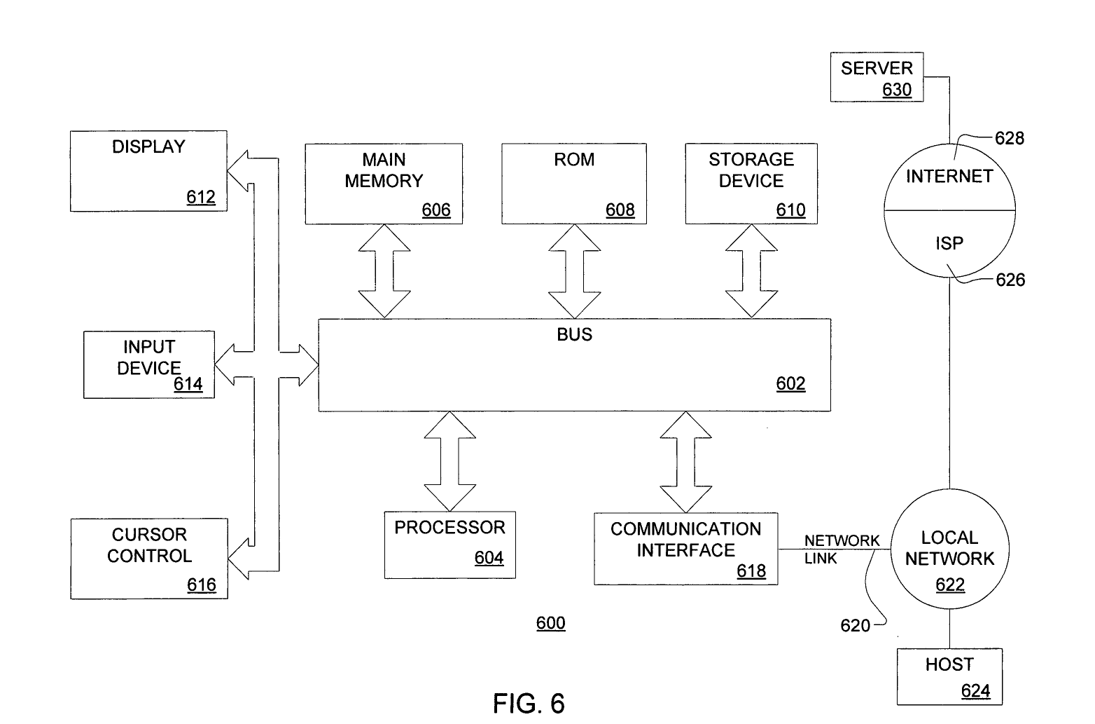

Referring now to FIG. 6, a block diagram illustrating the computer system 600 upon which an embodiment of the invention may be implemented is shown. Computer system 600 includes a bus 602 or other communication mechanism for communicating information, and a processor 604 coupled with bus 602 for processing information. Computer system 600 also includes a main memory 606, such as a random access memory (RAM) or other dynamic storage device, coupled to bus 602 for storing information and instructions to be executed by processor 604. Main memory 606 also may be used for storing temporary variables or other intermediate information during execution of instructions to be executed by processor 604. Computer system 600 further includes a read only memory (ROM) 608 or other static storage device coupled to bus 602 for storing static information and instructions for processor 604. A storage device 610, such as a magnetic disk or optical disk, is provided and coupled to bus 602 for storing information and instructions.

Computer system 600 may be coupled via bus 602 to a display 612, such as a cathode ray tube (CRT), for displaying information to a computer user. An input device 614, including alphanumeric and other keys, is coupled to bus 602 for communicating information and command selections to processor 604. Another type of user input device is cursor control 616, such as a mouse, a trackball, or cursor direction keys for communicating direction information and command selections to processor 604 and for controlling cursor movement on display 612. This input device typically has two degrees of freedom in two axes, a first axis (e.g., x) and a second axis (e.g., y), that allows the device to specify positions on a plane.

The invention is related to the use of computer system 600 for determining the genetic relationship of a customer to at least one group of famous individuals. According to one embodiment of the invention, determining the genetic relationship of a customer to at least one group of famous individuals is provided by computer system 600 in response to processor 604 executing one or more sequences of one or more instructions contained in main memory 606. Such instructions may be read into main memory 606 from another computer-readable medium, such as storage device 610. Execution of the sequences of instructions contained in main memory 606 causes processor 604 to perform the process steps described herein. One or more processors in a multi-processing arrangement may also be employed to execute the sequences of instructions contained in main memory 606. In alternative embodiments, hard-wired circuitry may be used in place of or in combination with software instructions to implement the invention. Thus, embodiments of the invention are not limited to any specific combination of hardware circuitry and software.

The term “computer-readable medium” as used herein refers to any medium that participates in providing instructions to processor 604 for execution. Such a medium may take many forms, including but not limited to, non-volatile media, volatile media, and transmission media. Non-volatile media include, for example, optical or magnetic disks, such as storage device 610. Volatile media include dynamic memory, such as main memory 606. Transmission media include coaxial cables, copper wire and fiber optics, including the wires that comprise bus 602. Transmission media can also take the form of acoustic or light waves, such as those generated during radio wave and infrared data communications.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, an EPROM, a FLASH-EPROM, any other memory chip or cartridge, a carrier wave as described hereinafter, or any other medium from which a computer can read.

Various forms of computer readable media may be involved in carrying one or more sequences of one or more instructions to processor 604 for execution. For example, the instructions may initially be carried on a magnetic disk of a remote computer. The remote computer can load the instructions into its dynamic memory and send the instructions over a telephone line using a modem. A modem local to computer system 600 can receive the data on the telephone line and use an infrared transmitter to convert the data to an infrared signal. An infrared detector coupled to bus 602 can receive the data carried in the infrared signal and place the data on bus 602. Bus 602 carries the data to main memory 606, from which processor 604 retrieves and executes the instructions. The instructions received by main memory 606 may optionally be stored on storage device 610 either before or after execution by processor 604.

Computer system 600 also includes a communication interface coupled to bus 602. Communication interface 618 provides a two-way data communication coupling to a network link 620 that is connected to a local network 622. For example, communication interface 618 may be an integrated services digital network (ISDN) card or a modem to provide a data communication connection to a corresponding type of telephone line. As another example, communication interface 618 may be a local area network (LAN) card to provide a data communication connection to a compatible LAN. Wireless links may also be implemented. In any such implementation, communication interface 618 sends and receives electrical, electromagnetic or optical signals that carry digital data streams representing various types of information.

Network link 620 (and shown as 106 in FIG. 1) typically provides data communication through one or more networks to other data devices. For example, network link 620 may provide a connection through local network 622 to a host computer 624 or to data equipment operated by an Internet Service Provider (ISP) 626. ISP 626 in turn provides data communication services through the worldwide packet data communication network now commonly referred to as the “Internet” 628. Local network 622 and Internet 628 both use electrical, electromagnetic or optical signals that carry digital data streams. The signals through the various networks and the signals on network link 620 and through communication interface 618, which carry the digital data to and from computer system 600, are exemplary forms of carrier waves transporting the information.

Computer system 600 can send messages and receive data, including program code, through the network(s), network link 620 and communication interface 618. In the Internet example, a server 630 might transmit a requested code for an application program through Internet 628, ISP 626, local network 622 and communication interface 618. In accordance with the invention, one such downloaded application provides for determining the genetic relationship of a customer to at least one group of famous individuals as described herein.

The received code may be executed by processor 604 as it is received, and/or stored in storage device 610, or other non-volatile storage for later execution. In this manner, computer system 600 may obtain application code in the form of a carrier wave.

Although the embodiments and description herein describe determining the genetic relationship between a group of famous persons and a customer, the methods described herein can also be used to determine the genetic relationship between an animal and any group of famous animals. For example, groups of famous animals include, without limit, Triple Crown winners, champion racehorses, kennel club champions, Westminster Kennel Club Dog Show winners, champion cats, Hollywood cats, Hollywood dogs and Hollywood horses.

A number of embodiments of the present invention have been described. Nevertheless, it will be understood that various modifications may be made without departing from the spirit and scope of the invention. Accordingly, although the present invention has been described with reference to certain preferred embodiments thereof, other versions are readily apparent to those of ordinary skill in the art, and therefore, it is to be understood that the invention is not limited by the specific illustrated embodiment, but only by the scope of the appended claims.

What is claimed is:

1. A method for quantifying the genetic relationship of a customer to at least one individual of at least one group of famous individuals, said method comprising the steps of:
receiving genetic information and famous individual group selection criteria from a customer;
calculating genetic distance of said customer to at least one individual within said group; and
reporting said results to said customer.

2. The method as claimed in claim 1, wherein said genetic information is a biological sample provided by said consumer.

3. The method as claimed in claim 1, wherein said genetic information is data provided by said consumer.

4. The method as claimed in claim 1, wherein said calculating said genetic distance of the customer further comprising determining the proportion of a control population that is more closely or less closely genetically related to said at least one individual within said group as compared with said customer.

5. The method as claimed in claim 4, wherein said reporting said results to said customer includes ranking the order of most closely genetically related famous individual s to said customer.

6. A system for quantifying the genetic relationship of a customer to at least one individual of at least one group of famous individuals, the system comprising one or more computers operably programmed and configured to:
receive genetic information and selection input, said input selecting at least one group of famous individuals;
process said genetic information and selection input to calculate a first genetic distance value between said genetic information received and each famous individual within said group; and
display a result of said calculation, whereby said result is a list of the famous individuals and a representation of said first genetic distance value.

7. The system of claim 6 wherein displaying a result further comprising ranking the order of said genetic distance.

8. The system of claim 6 wherein the result of said calculation is displayed on an Internet website.

9. The system of claim 6 wherein the result of said calculation is displayed via computer monitor.

10. The system of claim 6 wherein processing said genetic information further comprising calculating a second genetic distance value between a control population and each famous individual within said group and comparing said second genetic distance values with said first genetic distance value to yield a relationship value.
